(19) United States
(12) Patent Application Publication
(10) Pub. No.: US 2007/0166259 A1
(43) Pub. Date: Jul. 19, 2007

(54) Cosmetic composition comprising a tensioning agent and a particular block ethylenic polymer

(75) Inventors: Marco Vicic, Bry sur Marne (FR); Guillaume Cassin, Villebon sur Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/573,579

(22) PCT Filed: Sep. 22, 2004
(86) PCT No.: PCT/EP04/52270

§ 371 Date: Dec. 26, 2006

Related U.S. Application Data
(60) Provisional application No. 60/508,313, filed on Oct. 6, 2003.

(30) Foreign Application Priority Data
Sep. 26, 2003 (FR) 0311338

(51) Int. Cl. A61K 8/81

(52) U.S. Cl. 424/70.11; 424/70.16

(57) ABSTRACT

The present invention relates to a cosmetic composition adapted for a topical application on the skin, especially an anti-wrinkle composition, comprising, in a medium compatible with the skin: at least one tensioning agent, the said tensioning agent being present in a content ranging from 0.01% to 20% relative to the total weight of the composition; and at least one non-elastomeric, water-in soluble film-forming linear block ethylenic polymer, the said polymer being present in a content ranging from 0.01% to 20% relative to the total weight of the composition. This composition makes it possible to give a remanent tensioning effect to the skin to which it is applied.

### TECHNICAL FIELD

The present invention relates to a cosmetic composition adapted to a topical application on the skin, especially an anti-wrinkle composition, comprising, in a medium compatible with the skin, a tensioning agent and a particular polymer capable of giving rise to remanence of the tensioning effect induced by the tensioning agent.

The present invention relates to the use of this particular polymer to improve the remanence of the tensioning effect afforded by a tensioning agent.

The present invention relates to the use of this particular polymer in a composition comprising a colloidal dispersion of mineral particles to prevent bleaching of the skin.

Finally, the present invention relates to a process for treating wrinkled skin by applying to the said skin a composition as defined above.

The general field of the invention is thus that of ageing of the skin.

In the course of ageing of the skin, different signs appear, reflected especially by a change in the structure and functions of the skin. One of these main signs is the appearance of fine lines and deep wrinkles, the size and number of which increase with age. The skin microrelief becomes less uniform and has an anisotropic nature.

### PRIOR ART

It is common practice to treat these signs of ageing with cosmetic compositions containing active agents capable of combating ageing, such as α-hydroxy acids, β-hydroxy acids and retinoids. These active agents act especially on wrinkles by removing the dead cells from the skin and by accelerating the process of cell renewal. However, these active agents have the drawback of being effective in treating wrinkles only after they have been applied for a certain amount of time, i.e. a time that may be from a few days to several weeks.

Now, the current needs are increasingly tending towards the production of compositions for obtaining an immediate effect, leading rapidly to smoothing-out of the wrinkles and/or fine lines and to the disappearance, even temporary, of fatigue marks. Such compositions are compositions comprising tensioning agents. It is pointed out that the term “tensioning agent” means compounds capable of having a tensioning effect, i.e. compounds that can make the skin taut and bring about a reduction in or even the immediate disappearance therefrom of wrinkles and fine lines.

These tensioning agents may especially be polymers of natural or synthetic origin in aqueous dispersion, capable of forming a film that causes shrinkage of the stratum corneum, the superficial horny layer of the epidermis. The cosmetic or dermatological use of such polymer systems to attenuate the effects of ageing of the skin is described in patent application FR-A-2 758 083 [1].

However, these tensioning polymer systems occasionally give a sensation of discomfort to certain users, especially to those with fragile skin. In addition, the tensioning effect they afford is not very long-lasting, since the film formed on the skin has a tendency to crack as a result of the facial expressions. The reason for this is that these tensioning agents form a relatively rigid and inflexible film on the skin.

The Applicant has found, surprisingly, that the use of particular polymers in combination with a tensioning agent in a cosmetic composition makes it possible to obtain films that have a long-lasting tensioning effect, the said films being flexible and deformable from a mechanical point of view.

In addition, the Applicant has noted that these particular polymers furthermore have the property of avoiding bleaching of the skin following the application thereto of compositions comprising, as tensioning agents, colloidal dispersions of mineral particles, in particular of silica.

### DESCRIPTION OF THE INVENTION

Thus, according to a first subject, the invention relates to a cosmetic composition adapted for an application on the skin, such as the face, especially an anti-wrinkle composition, comprising, in a medium compatible with the skin:
at least one tensioning agent, the said tensioning agent being present in a content ranging from 0.01% to 20% relative to the total weight of the composition, and
at least one non-elastomeric, water-insoluble film-forming linear block ethylenic polymer, the said polymer being present in a content ranging from 0.01% to 20% relative to the total weight of the composition.

The use of polymers as defined above in combination with a tensioning agent makes it possible to give the composition in which they are included a remanent tensioning effect, i.e. a tensioning effect that has a certain durability over time, the polymer acting as reinforcement for the tensioning film. A test directed towards demonstrating this remanence property of such a combination is described in the experimental section of this description. It is pointed out that this remanence is justified in the context of this invention by the improvement in the mechanical properties of the tensioning film.

Before going into further detail in the description, the following definitions are given.

According to the invention, the term “tensioning agent” means any agent that produces, at a concentration of 7% in water, a shrinkage of isolated stratum corneum, measured using an extensometer, of more than 1% and preferably of more than 1.5% at 30° C. under a relative humidity of 40%.

The protocol for determining the stratum corneum shrinkage is as follows:

The tensioning power of the tensioning agents described in the present document was measured using an extensometer.

The principle of the method consists in measuring the length of a sample of stratum corneum isolated from human skin derived from a surgical operation, before and after treatment with the test compositions.

To do this, the sample is placed between the two jaws of the machine, one of which is fixed and the other mobile, under an atmosphere at 30° C. and 40% relative humidity. A tension is exerted on the sample, and the curve of the force (in grams) as a function of the length (in millimetres) is recorded, zero length corresponding to contact between the two jaws of the machine. The tangent to the curve in its linear region is then plotted. The intersection of this tangent with the x-axis corresponds to the apparent length L₀ of the sample at zero force. The sample is then relaxed, after which 2 mg/cm² of the test composition (solution containing 7% of the tensioning agent under consideration) are applied to the stratum corneum. After drying for 15 minutes, the above steps are repeated to determine the length of L₁, of the sample after treatment. The percentage of shrinkage is defined by: % shrinkage =100×(L₁−L₀)/L₀. To characterize a tensioning effect, this percentage must be negative, and the tensioning effect is proportionately greater the higher the absolute value of the percentage of shrinkage.

In the text hereinabove and hereinbelow, the term “ethylenic polymer” means a polymer obtained by polymerization of monomers comprising an ethylenic unsaturation.

In the text hereinabove and hereinbelow, the term “block polymer” means a polymer comprising at least two different blocks and preferably at least three different blocks.

The polymer according to the invention is a polymer of linear structure. In contrast, a polymer of non-linear structure is, for example, a polymer of branched structure, of starburst or grafted form, or the like.

The term “film-forming polymer” means a polymer capable of forming, by itself or in the presence of a film-forming auxiliary agent, a continuous film that adheres to a support, especially to keratin materials.

The term “non-elastomeric polymer” means a polymer which, when it is subjected to a constraint intended to stretch it (for example by 30% relative to its initial length), does not return to a length substantially identical to its initial length when the constraint ceases.

More specifically, the term “non-elastomeric polymer” denotes a polymer with an instantaneous recovery Rᵢ<50% and a delayed recovery R₂ₕ<70% after having been subjected to a 30% elongation.

Preferably, Rᵢ is <30% and R₂ₕ<50%.

More specifically, the non-elastomeric nature of the polymer is determined according to the following protocol:

A polymer film is prepared by pouring a solution of the polymer in a Teflon-coated mould, followed by drying for 7 days in an environment conditioned at 23±5° C. and 50±10% relative humidity.

A film about 100 μm thick is thus obtained, from which are cut rectangular specimens (for example using a punch) 15 mm wide and 80 mm long.

This sample is subjected to a tensile stress using a machine sold under the reference Zwick, under the same temperature and humidity conditions as for the drying.

The specimens are pulled at a speed of 50 mm/min and the distance between the jaws is 50 mm, which corresponds to the initial length (l₀) of the specimen.

The instantaneous recovery Rᵢ is determined in the following manner:
the specimen is pulled by 30% (εₘₐₓ), i.e. about 0.3 times its initial length (l₀)
the constraint is released by applying a return speed equal to the tensile speed, i.e. 50 mm/min, and the residual elongation of the specimen is measured as a percentage, after returning to zero constraint (εᵢ).

The instantaneous recovery Rᵢ (in %) is determined by the following formula:
Rᵢ=((εₘₐₓ−εᵢ) /εₘₐₓ)×100

To determine the delayed recovery, the percentage residual elongation of the specimen (ε₂ₕ) 2 hours after returning to zero constraint is measured after two hours.

The percentage delayed recovery R₂ₕ (in %) is given by the following formula:
R₂ₕ=((εₘₐₓ−ε₂ₕ)/εₘₐₓ)×100

As a guide, a polymer according to one preferred embodiment has an instantaneous recovery Rᵢ of 10% and a delayed recovery R₂ₕ of 30%.

The term “water-insoluble polymer” means that the polymer is not soluble in water or in a mixture of water and of linear or branched lower alcohols containing from 2 to 5 carbon atoms, for instance ethanol, isopropanol or n-propanol, without pH modification, at an active material content of at least 1% by weight, at room temperature (25° C.).

As mentioned above, the composition according to the invention comprises at least one tensioning agent. The said agent is included in the composition in a content ranging from 0.01% to 20% and preferably from 1% to 10% relative to the total weight of the composition.

The said tensioning agent may especially be chosen from:
a) synthetic polymers;
b) polymers of natural origin;
c) mixed silicates;
d) wax microparticles;
e) colloidal particles of mineral fillers;
and mixtures thereof.
   a) Synthetic Polymers

Synthetic polymers that may be used as tensioning agent may be chosen from:
polyurethane polymers and copolymers;
acrylic polymers and copolymers;
sulfonated isophthalic acid polymers;
grafted silicone polymers;
water-soluble or water-dispersible polymers comprising water-soluble or water-dispersible units and units with an LCST;
and mixtures thereof.

The polyurethane copolymers, the acrylic copolymers and the other synthetic polymers according to the invention may be chosen especially from polycondensates, hybrid polymers and interpenetrated polymer networks (IPNs).

For the purposes of the present invention, the expression “interpenetrated polymer network” means a blend of two interlaced polymers, obtained by simultaneous polymerization and/or crosslinking of two types of monomer, the blend obtained having a single glass transition temperature.

Examples of IPNs that are suitable for use in the present invention, and also the process for preparing them, are described in patents U.S. Pat. No. 6,139,322 [2] and U.S. Pat. No. 6,465,001 [3], for example.

Preferably, the IPN according to the invention comprises at least one polyacrylic polymer and more preferably also comprises at least one polyurethane or one copolymer of vinylidene fluoride and of hexafluoropropylene.

According to one preferred form, the IPN according to the invention comprises a polyurethane polymer and a polyacrylic polymer. Such IPNs are especially those of the Hybridur series that are commercially available from the company Air Products.

An IPN that is particularly preferred is in the form of an aqueous dispersion of particles with a weight-average size of between 90 and 110 nm and a number-average size of about 80 nm. This IPN preferably has a glass transition temperature, Tg, ranging from about −60° C. to +100° C. An IPN of this type is sold especially by the company Air Products under the trade name Hybridur X-01602. Another IPN that is suitable for use in the present invention is referenced Hybridur X18693-21.

Other IPNs that are suitable for use in the present invention comprise IPNs consisting of a blend of a polyurethane with a copolymer of vinylidene fluoride and of hexafluoropropylene. These IPNs may be prepared especially as described in patent U.S. Pat. No. 5,349,003 [4]. As a variant, they are commercially available in the form of a colloidal dispersion in water, in a ratio of the fluorinated copolymer to the acrylic polymer of between 70:30 and 75:25, under the trade names Kynar RC-10, 147 and Kynar RC-10, 151 from the company Atofina.

Examples of grafted silicone polymers are given in patent application EP-1 038 519 [5], which is incorporated herein by reference. A preferred example of a grafted silicone polymer is polysilicone-8 (CTFA name), which is a polydimethylsiloxane onto which are grafted, via a linking chain of thiopropylene type, mixed polymer units of the poly(meth)acrylic acid type and of the poly(meth)acrylate type. A polymer of this type is available especially under the trade name VS 80 (at 10% in water) or LO 21 (in pulverulent form) from the company 3M. It is a copolymer of polydimethylsiloxane containing propylthio groups, of methyl acrylate, of methyl methacrylate and of methacrylic acid.

The above-mentioned synthetic polymers may be in the form of latices. As suitable latices that may be used according to the invention as tensioning agent, mention may be made especially of polyester-polyurethane and polyether-polyurethane dispersions such as those sold under the names “Avalure UR410” and “Avalure UR460” by the company Noveon, and under the names “Neorez R974”, “Neorez R981” and “Neorez R970”, and also acrylic copolymer dispersions such as those sold under the name “Neocryl XK-90” by the company Avecia.

It is also possible according to the invention to use water-soluble or water-dispersible polymers comprising water-soluble or water-dispersible units and comprising units with an LCST, the said units with an LCST having, in particular, a demixing temperature in water of from 5 to 40° C. at a mass concentration of 1%. This type of polymer is described more fully in patent application FR 2 819 429 [6].

b) Polymers of Natural Origin

The polymers of natural origin that may be used as tensioning agent may be chosen from:
plant proteins and plant protein hydrolysates;
polysaccharides of plant origin optionally in the form of microgels, such as starch;
latices of plant origin;
and mixtures thereof.

Examples of plant proteins and plant protein hydrolysates that may be used as tensioning agents according to the invention consist of proteins and protein hydrolysates from maize, rye, Triticum aestivum wheat, buckwheat, sesame, spelt, pea, bean, lentil, soybean and lupin.

Polysaccharides that are suitable for formulating the compositions according to the invention are any polysaccharide of natural origin capable of forming heat-reversible or crosslinked gels and also solutions. The term “heat-reversible” means that the gel state of these polymer solutions is obtained reversibly once the solution has been cooled below the characteristic gelation temperature of the polysac-charide used.

A first family of polysaccharides of natural origin that may be used in the present invention consists of carrageenans and most particularly kappa-carrageenan and iota-carrageenan. These are linear polysaccharides present in certain red algae. They consist of alternating β-1,3 and α-1,4 galactose residues, many galactose residues possibly being sulfated. This family of polysaccharides is described in the book “Food Gels” edited by Peter Harris, Elsevier 1989, chapter 3 [7]. Another family of polysaccharides that may be used consists of the Agars. These are also polymers extracted from red algae and they consist of alternating 1,4-L-galactose and 1,3-D-galactose residues. This family of polysaccharides is also described in chapter 1 of the book “Food Gels” [8] mentioned previously. A third family of polysaccharides consists of polysaccharides of bacterial origin known as gellans. These are polysaccharides consisting of an alternation of glucose, glucuronic acid and rhamnose residues. These gellans are described in particular in chapter 6 of the book “Food Gels” [9] mentioned previously. In the case of polysaccharides forming gels of crosslinked type, in particular induced by adding salts, mention will be made of polysaccharides belonging to the family of alginates and pectins.

Mention may also be made of chitosans and derivatives thereof, pullulans and derivatives thereof, and also mixtures of oppositely charged polymers that form complexes by means of electrostatic interactions.

The tensioning polysaccharides may be present in the form of microgels as described in FR 2 829 025 [10].

One particularly advantageous category of polysaccharides that may be used according to the invention consists of starch and derivatives thereof.

Starch is a natural product that is well known to those skilled in the art. It consists of a linear or branched polymer or polymer mixture consisting of α-D-glucopyranosyl units. Starch is described in particular in “Kirk-Othmer Encyclopedia of Chemical Technology, 3rd edition, volume 21, pp. 492-507, Wiley Interscience, 1983”[11].

The starch used according to the present invention may be of any origin: rice, maize, potato, cassava, pea, Triticum aestivum wheat, oat, etc. It may be natural or optionally modified by treatment such as crosslinking, acetylation or oxidation. It may optionally be grafted.

As starches that may be used in the composition according to the invention, mention may be made, for example, of the starch sold by the company Lambert-Riviere under the name Remi Dri.

c) Mixed Silicates

Another class of tensioning agents that may be used according to the invention consists of mixed silicates. This expression refers to any silicate of natural or synthetic origin containing several types of cation chosen from alkali metals (for example Na, Li or K) or alkaline-earth metals (for example Be, Mg or Ca) and transition metals.

Phyllosilicates are preferably used, i.e. silicates having a structure in which the SiO₄ tetrahedra are organized as leaflets between which are enclosed the metal cations.

Another family of silicates that is particularly preferred as tensioning agents is the laponite family. Laponites are magnesium lithium sodium silicates with a layered structure similar to that of montmorillonites. Laponite is the synthetic form of the natural mineral known as “hectorite”. Use may be made, for example, of the laponite sold under the name Laponite XLS or Laponite XLG by the company Rockwood.

d) Wax Microdispersions

Yet another class of tensioning agents that may be used in the present invention consists of wax microparticles. These are particles with a diameter generally less than 5 μm or, better still, 0.5 μm, and consisting essentially of a wax or a mixture of waxes chosen, for example, from carnauba wax, candelilla wax and esparto grass wax. The melting point of the wax or of the mixture of waxes is preferably between 50° C. and 150° C.

e) Colloidal Particles of Mineral Fillers

As another variant, it is also possible to use colloidal particles of mineral fillers as tensioning agent according to the invention. The term “colloidal particles” means generally colloidal particles dispersed in an aqueous, aqueous-alcoholic or alcoholic medium, with a numerical mean diameter of between 0.1 and 100 nm and preferably between 3 and 30 nm.

Examples of mineral fillers include: silica, cerium oxide, zirconium oxide, alumina, calcium carbonate, barium sulfate, calcium sulfate, zinc oxide and titanium dioxide. A mineral filler that is particularly preferred is silica. Colloidal silica particles are especially available in the form of an aqueous dispersion of colloidal silica from the company Catalysts & Chemicals under the trade names Cosmo S-40 and Cosmo S-50.

One particular example of colloidal particles of mineral fillers may be silica-alumina composite colloidal particles. The term “silica-alumina composite” means silica particles in which the aluminium atoms have been partially replaced with silicon atoms. These particles are in the form of aqueous dispersions and have no thickening properties in water, alcohol, oil or any other solvent. At a concentration of greater than or equal to 15% by weight in water, the viscosity of the solutions thus obtained is less than 0.05 Pa.s for a shear rate equal to 10 s^{l}. The measurements are performed at 25° C. using a Haake RS150 RheoStress rheometer in cone-plate configuration, the statistics of the measuring cone being: diameter: 60 mm and angle: 2°.

At pH 7, the silica-alumina composite colloidal particles according to the invention have a zeta potential of less than −20 mV and preferably less than −25 mV. The measurements are performed at 25° C. using a Delsa 440SX machine from Coulter Scientific Instrument.

As silica-alumina composite colloidal particles that may be used in the compositions according to the invention, examples that may be mentioned include those sold by the company Grace under the names Ludox AM, Ludox HSA and Ludox TMA.

As mentioned previously, the composition comprises a non-elastomeric, water-insoluble film-forming linear block ethylenic polymer.

This particular polymer in combination with a tensioning agent gives the composition into which it is incorporated a remanent tensioning effect, by virtue of its capacity to reinforce the tensioning film while at the same time giving it flexibility properties. This remanence is quantified according to the invention by measuring the improvement in the mechanical properties of the tensioning film (more particularly by measuring the improvement in the breaking strength), as will be explained in the protocol given in the experimental section of this description.

This polymer is present in the composition in a content ranging from 0.01% to 20% and preferably from 1% to 10% relative to the total weight of the composition, the said polymer preferably being present at most in an amount equal to that of the tensioning agent.

This type of polymer according to the invention advantageously comprises at least one first block and at least one second block that are mutually incompatible and that have different glass transition temperatures (Tg), the said first and second blocks being connected together via an intermediate segment comprising at least one constituent monomer of the first block and at least one constituent monomer of the second block, the said polymer having a polydispersity index I of greater than 2.

The Tg values of the first and second blocks are theoretical Tg values calculated according to Fox's law.

The expression “mutually incompatible blocks” means that the blend formed from the polymer corresponding to the first block and from the polymer corresponding to the second block is not miscible in the polymerization solvent that is in majority amount by weight for the block polymer, at room temperature (25° C.) and atmospheric pressure (10⁵ Pa), for a polymer blend content of greater than or equal to 5% by weight, relative to the total weight of the blend (polymers and solvent), it being understood that:

i) the said polymers are present in the blend in a content such that the respective weight ratio ranges from 10/90 to 90/10, and that

ii) each of the polymers corresponding to the first and second blocks has an average (weight-average or number-average) molecular mass equal to that of the block polymer ±15%.

In the case of a mixture of polymerization solvents, should two or more solvents be present in identical mass proportions, the said polymer blend is immiscible in at least one of them.

Needless to say, in the case of a polymerization performed in only one solvent, this solvent constitutes the solvent that is in majority amount.

The intermediate segment, which is a block comprising at least one constituent monomer of the first block and at least one constituent monomer of the second block, allows these blocks to be “compatibilized”.

It is pointed out that, in the text hereinabove and hereinbelow, the terms “first block” and “second block” do not in any way condition the order of the said blocks in the structure of the polymer.

The polydispersity index I of the polymer is equal to the ratio of the weight-average mass Mw to the number-average mass Mn.

The weight-average molar mass (Mw) and number-average molar mass (Mn) are determined by gel permeation liquid chromatography (THF solvent, calibration curve established with linear polystyrene standards, refractometric detector).

The weight-average mass (Mw) of the polymer used in the composition according to the invention is preferably less than or equal to 300 000; it ranges, for example, from 35 000 to 200 000 and better still from 45 000 to 150 000.

The number-average mass (Mn) of the polymer used in the composition according to the invention is preferably less than or equal to 70 000; it ranges, for example, from 10 000 to 60 000 and better still from 12 000 to 50 000.

Preferably the polydispersity index of the polymer used in the composition according to the invention is greater than 2, for example greater than 2 and less than or equal to 9, preferably greater than or equal to 2.5, for example ranging from 2.5 to 8, and better still greater than or equal to 2.8, especially from 2.8 to 6.

Each block of the polymer used in the composition according to the invention is derived from one type of monomer or from several different types of monomer.

This means that each block may consist of a homopolymer or a copolymer; this copolymer constituting the block may in turn be random or alternating.

Advantageously, the intermediate block comprising at least one constituent monomer of the first block and at least one constituent monomer of the second block of the polymer is a random polymer.

Preferably, the intermediate block is derived essentially from constituent monomers of the first block and of the second block.

The term “essentially” means at least 85%, preferably at least 90%, better still 95% and even better still 100%.

Advantageously, the intermediate block has a glass transition temperature Tg that is between the glass transition temperatures of the first and second blocks.

According to the invention, the first and second blocks have different glass transition temperatures.

The glass transition temperatures indicated for the first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:
1/Tg=Eᵢ ( ω̅ᵢ/Tgᵢ),
Eᵢ representing the sum symbol in i, ω̅ᵢ being the mass fraction of the monomer i in the block under consideration and Tgᵢ being the glass transition temperature of the homopolymer of the monomer i.

Unless otherwise indicated, the Tg values indicated for the first and second blocks in the present patent application are theoretical Tg values.

The difference between the glass transition temperatures of the first and second blocks is generally greater than 20° C., preferably greater than 30° C. and better still greater than 40° C.

In particular, the first block may be chosen from:
a) a block with a Tg of greater than or equal to 40° C.,
b) a block with a Tg of less than or equal to 20° C.,
c) a block with a Tg of between 20 and 40° C., and the second block being chosen from a category a),
b) or c) different from the first block.

In the present invention, the expression: “between . . . and . . . ” is intended to denote a range of values for which the limits mentioned are excluded, and “from . . . to . . . ” and “ranging from . . . to . . . ” are intended to denote a range of values for which the limits are included.

a) Block With a Tg of Greater Than or Equal To 40° C.

The block with a Tg of greater than or equal to 40° C. has, for example, a Tg ranging from 40 to 150° C., preferably greater than or equal to 50° C., for example ranging from 50° C. to 120° C. and better still greater than or equal to 60° C., for example ranging from 60° C. to 120° C.

The block with a Tg of greater than or equal to 40° C. may be a homopolymer or a copolymer.

In the case where this block is a homopolymer, it is derived from monomers which are such that the homopolymers prepared from these monomers have glass transition temperatures of greater than or equal to 40° C. This first block may be a homopolymer consisting of only one type of monomer (for which the Tg of the corresponding homopolymer is greater than or equal to 40° C.).

In the case where the first block is a copolymer, it may be totally or partially derived from one or more monomers, the nature and concentration of which are chosen such that the Tg of the resulting copolymer is greater than or equal to 40° C. The copolymer may comprise, for example:
monomers which are such that the homopolymers prepared from these monomers have Tg values of greater than or equal to 40° C., for example a Tg ranging from 40 to 150° C., preferably greater than or equal to 50° C., for example ranging from 50° C. to 120° C. and better still greater than or equal to 60° C., for example ranging from 60° C. to 120° C., and
monomers which are such that the homopolymers prepared from these monomers have Tg values of less than 40° C., chosen from monomers with a Tg of between 20 and 40° C. and/or monomers with a Tg of less than or equal to 20° C., for example a Tg ranging from −100 to 20° C., preferably less than 15° C., especially ranging from −80° C. to 15° C. and better still less than 10° C., for example ranging from −50° C. to 0° C., as described later.

The monomers whose homopolymers have a glass transition temperature of greater than or equal to 40° C. are chosen, preferably, from the following monomers, also known as the main monomers:
methacrylates of formula CH₂=C(CH₃)—COOR₁ in which R₁ represents a linear or branched unsubstituted alkyl group containing from 1 to 4 carbon atoms, such as a methyl, ethyl, propyl or isobutyl group or R₁ represents a C₄ to C₁₂ cycloalkyl group,
acrylates of formula CH₂=CH—COOR₂ in which R₂ represents a C₄ to C₁₂ cycloalkyl group such as an isobornyl, or a tert-butyl group,
(meth)acrylamides of formula: 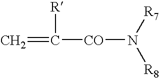
   in which R₇ and R₈, which may be identical or different, each represent a hydrogen atom or a linear or branched C₁ to C₁₂ alkyl group such as an n-butyl, t-butyl, isopropyl, isohexyl, isooctyl or isononyl group; or R₇ represents H and R₈ represents a 1,1-dimethyl-3-oxobutyl group, and R′ denotes H or methyl.

Examples of monomers that may be mentioned include N-butylacrylamide, N-t-butylacrylamide, N-iso-propylacrylamide, N,N-dimethylacrylamide and N,N-dibut-ylacrylamide,
and mixtures thereof.

Main monomers that are particularly preferred are methyl methacrylate, isobutyl (meth)acrylate, isobornyl (meth)acrylate, and mixtures thereof.

b) Block With a Tg of Less Than or Equal to 20° C.

The block with a Tg of less than or equal to 20° C. has, for example, a Tg ranging from −100 to 20° C., preferably less than or equal to 15° C., especially ranging from −80° C. to 15° C. and better still less than or equal to 10° C., for example ranging from −50° C. to 0° C.

The block with a Tg of less than or equal to 20° C. may be a homopolymer or a copolymer.

In the case where this block is a homopolymer, it is derived from monomers which are such that the homopolymers prepared from these monomers have glass transition temperatures of less than or equal to 20° C. This second block may be a homopolymer consisting of only one type of monomer (for which the Tg of the corresponding homopolymer is less than or equal to 20° C.).

In the case where the block with a Tg of less than or equal to 20° C. is a copolymer, it may be totally or partially derived from one or more monomers, the nature and concentration of which are chosen such that the Tg of the resulting copolymer is less than or equal to 20° C.

It may comprise, for example
one or more monomers whose corresponding homopolymer has a Tg of less than or equal to 20° C., for example a Tg ranging from −100° C. to 20° C., preferably less than 15° C., especially ranging from −80° C. to 15° C. and better still less than 10° C., for example ranging from −50° C. to 0° C., and
one or more monomers whose corresponding homopolymer has a Tg of greater than 20° C., such as monomers with a Tg of greater than or equal to 40° C., for example a Tg ranging from 40 to 150° C., preferably greater than or equal to 50° C., for example ranging from 50° C. to 120° C. and better still greater than or equal to 60° C., for example ranging from 60° C. to 120° C. and/or monomers with a Tg of between 20 and 40° C., as described above.

Preferably, the block with a Tg of less than or equal to 20° C. is a homopolymer.

The monomers whose homopolymer has a Tg of less than or equal to 20° C. are preferably chosen from the following monomers, or main monomers:
acrylates of formula CH₂=CHCOOR₃, R₃ representing a linear or branched C₁, to C₁₂ alkyl group, with the exception of the tert-butyl group, in. which one or more hetero atoms chosen from O, N and S is (are) optionally intercalated, the said alkyl group also possibly being optionally substituted with one or more substituents chosen from hydroxyl groups and halogen atoms (Cl, Br, I and F), or R₃ represents an (alkyl group C₁-C₁₂)-O-POE (POE denoting polyoxyethylene with repetition of the oxyethylene group from 5 to 30 times), such as a methoxy-POE group, or R₃ represents a polyoxyethylene group comprising from 5 to 30 ethylene oxide units;
methacrylates of formula CH₂=C(CH₃)-COOR₄, R₄ representing a linear or branched C₆ to C₁₂ alkyl group, in which one or more hetero atoms chosen from O, N and S is (are) optionally intercalated, the said alkyl group also possibly being optionally substituted with one or more substituents chosen from hydroxyl groups and halogen atoms (Cl, Br, I or F);
vinyl esters of formula R₅—CO—O—CH═CH₂ in which R₅ represents a linear or branched C₄ to C₁₂ alkyl group;
C₄ to C₁₂ alkyl vinyl ethers, such as butyl vinyl ether and lauryl vinyl ether;
N-(C₄ to C₁₂-alkyl) acrylamides, such as N-octylacryl-amide;
and mixtures thereof.

The main monomers that are particularly preferred for the block with a Tg of less than or equal to 20° C. are alkyl acrylates in which the alkyl chain contains from 1 to 10 carbon atoms, with the exception of the tert-butyl group, such as methyl acrylate, isobutyl acrylate and 2-ethylhexyl acrylate, and mixtures thereof.

Preferably, the proportion of the second block with a Tg of less than or equal to 20° C. ranges from 10% to 85%, better still from 20% to 70% and even better still from 20% to 50% by weight of the polymer.

c) Block With a Tg of Between 20 and 40° C.

The block with a Tg of between 20 and 40° C. may be a homopolymer or a copolymer.

In the case where this block is a homopolymer, it is derived from monomers (or main monomers) which are such that the homopolymers prepared from these monomers have glass transition temperatures of between 20 and 40° C. This first block may be a homopolymer, consisting of only one type of monomer (for which the Tg of the corresponding homopolymer ranges from 20° C. to 40° C.).

The monomers whose homopolymer has a glass transition temperature of between 20 and 40° C. are preferably chosen from n-butyl methacrylate, cyclodecyl acrylate, neopentyl acrylate and isodecylacrylamide, and mixtures thereof.

In the case where the block with a Tg of between 20 and 40° C. is a copolymer, it is totally or partially derived from one or more monomers (or main monomers) whose nature and concentration are chosen such that the Tg of the resulting copolymer is between 20 and 40° C.

Advantageously, the block with a Tg of between 20 and 40° C. is a copolymer totally or partially derived from:
main monomers whose corresponding homopolymer has a Tg of greater than or equal to 40° C., for example a Tg ranging from 40° C. to 150° C., preferably greater than or equal to 50° C., for example ranging from 50 to 120° C. and better still greater than or equal to 60° C., for example ranging from 60° C. to 120° C., as described above, and/or
main monomers whose corresponding homopolymer has a Tg of less than or equal to 20° C., for example a Tg ranging from −100 to 20° C., preferably less than or equal to 15° C., especially ranging from −80° C. to 15° C. and better still less than or equal to 10° C., for example ranging from −50° C. to 0° C., as described above, the said monomers being chosen such that the Tg of the copolymer forming the first block is between 20 and 40° C.

Advantageously, each of the first and second blocks is totally derived from at least one monomer chosen from acrylic acid, acrylic acid esters, methacrylic acid and methacrylic acid esters, and mixtures thereof.

Preferably, the polymer according to the invention comprises no styrene or styrene derivatives, for instance methylstyrene, chlorostyrene or chloro-methylstyrene. In addition, according to one preferred mode of the invention, the polymer according to the invention is a non-silicone polymer, i.e. a polymer free of silicon atoms.

Each of the first and/or second blocks may comprise, in addition to the monomers indicated above, one or more other monomers known as additional monomers, which are different from the main monomers mentioned above.

The nature and amount of this or these additional monomer(s) are chosen such that the block in which they are present has the desired glass transition temperature.

This additional monomer is chosen, for example, from: a) hydrophilic monomers such as:
ethylenically unsaturated monomers comprising at least one carboxylic or sulfonic acid function, for instance: acrylic acid, methacrylic acid, crotonic acid, maleic anhydride, itaconic acid, fumaric acid, maleic acid, acrylamidopropanesulfonic acid, vinylbenzoic acid, vinylphosphoric acid, and salts thereof,
ethylenically unsaturated monomers comprising at least one hydroxyl function for instance 2-hydroxypropyl methacrylate, 2-hydroxyethyl methacrylate, 2-hydroxypropyl acrylate and 2-hydroxyethyl acrylate,
ethylenically unsaturated monomers comprising at least one tertiary amine function, for instance 2-vinylpyridine, 4-vinylpyridine, dimethylaminoethyl methacrylate, diethylaminoethyl methacrylate and dimethylaminopropylmethacrylamide, and salts thereof,
methacrylates of formula CH₂═C(CH₃)—COOR₆ in which R₆ represents a linear or branched alkyl group containing from 1 to 4 carbon atoms, such as a methyl, ethyl, propyl or isobutyl group, the said alkyl group being substituted with one or more substituents chosen from hydroxyl groups (for instance 2-hydroxypropyl methacrylate and 2-hydroxyethyl methacrylate) and halogen atoms (Cl, Br, I or F), such as trifluoroethyl methacrylate,
methacrylates of formula CH₂═C(CH₃)—COOR₉, R₉ representing a linear or branched C₆ to C₁₂ alkyl group in which one or more hetero atoms chosen from O, N and S is (are) optionally intercalated, the said alkyl group being substituted with one or more substituents chosen from hydroxyl groups and halogen atoms (Cl, Br, I or F);
acrylates of formula CH₂═CHCOOR₁₀, R₁₀ representing a linear or branched C₁ to C₁₂ alkyl group substituted with one or more substituents chosen from hydroxyl groups and halogen atoms (Cl, Br, I or F), such as 2-hydroxypropyl acrylate and 2-hydroxyethyl acrylate, or R₁₀ represents a (C₁ to C₁₂ alkyl)-O-POE (POE denoting polyoxyethylene with repetition of the oxyethylene unit 5 to 30 times), for example methoxy-POE, or R₁₀ represents a polyoxyethylenated group comprising from 5 to 30 ethylene oxide units b) ethylenically unsaturated monomers comprising one or more silicon atoms, such as methacryloxypropyltri-methoxysilane and methacryloxypropyltris(trimethyl-siloxy)silane,
and mixtures thereof.

Additional monomers that are particularly preferred are acrylic acid, methacrylic acid and trifluoroethyl methacrylate, and mixtures thereof.

This or these additional monomer(s), when they are present, generally represent(s) an amount of less than or equal to 30% by weight, for example from 1% to 30% by weight, preferably from 5% to 20% by weight and more preferably from 7% to 15% by weight, relative to the total weight of the first and/or second blocks.

The polymer according to the invention may be obtained by free-radical solution polymerization according to the following preparation process:
a portion of the polymerization solvent is introduced into a suitable reactor and heated until the adequate temperature for the polymerization is reached (typically between 60 and 120° C.),
once this temperature is reached, the constituent monomers of the first block are introduced in the presence of some of the polymerization initiator,
after a time T corresponding to a maximum degree of conversion of 90%, the constituent monomers of the second block and the rest of the initiator are introduced,
the mixture is left to react for a time T′ (ranging from 3 to 6 hours), after which the mixture is cooled to room temperature,
the polymer dissolved in the polymerization solvent is obtained.

The term “polymerization solvent” means a solvent or a mixture of solvents. The polymerization solvent may be chosen especially from ethyl acetate, butyl acetate, alcohols such as isopropanol or ethanol, and aliphatic alkanes such as isododecane, and mixtures thereof. Preferably, the polymerization solvent is a mixture of butyl acetate and isopropanol or isododecane.

The time T corresponds to a degree of conversion of 90%, i.e. to a percentage of consumed constituent monomers of the first block of 90%.

The polymerization temperature preferably ranges from 60 to 120° C. and preferentially from 80 to 100° C.

The polymerization initiator may be chosen from organic peroxides containing from 8 to 30 carbon atoms. An example that may be mentioned, for example, is 2, 5-bis(2-ethylhexanoylperoxy)-2, 5-dimethylhexane, sold under the reference Trigonoxe® 141 by the company Akzo Nobel.

### FIRST EMBODIMENT

According to a first embodiment, the polymer according to the invention comprises a first block with a Tg of greater than or equal to 40° C., as described above in a) and a second block with a Tg of less than or equal to 20° C., as described above in b).

Preferably, the first block with a Tg of greater than or equal to 40° C. is a copolymer derived from monomers which are such that the homopolymer prepared from these monomers has a glass transition temperature of greater than or equal to 40° C., such as the monomers described above.

Advantageously, the second block with a Tg of less than or equal to 20° C. is a homopolymer derived from monomers which are such that the homopolymer prepared from these monomers has a glass transition temperature of less than or equal to 20° C., such as the monomers described above.

Preferably, the proportion of the block with a Tg of greater than or equal to 40° C. ranges from 20% to 90%, better still from 30% to 80% and even better still from 50% to 70% by weight of the polymer.

Preferably, the proportion of the block with a Tg of less than or equal to 20° C. ranges from 5% to 75%, preferably from 15% to 50% and better still from 25% to 45% by weight of the polymer.

Thus, according to a first variant, the polymer according to the invention may comprise:
a first block with a Tg of greater than or equal to 40° C., for example with a Tg ranging from 70 to 110° C., which is a (methyl methacrylate/acrylic acid) copolymer,
a second block with a Tg of less than or equal to 20° C., for example ranging from 0 to 20° C., which is a methyl acrylate homopolymer, and
an intermediate block that is a (methyl methacrylate/acrylic acid/methyl acrylate) copolymer.

According to a second variant, the polymer according to the invention may comprise:
a first block with a Tg of greater than or equal to 40° C., for example ranging from 70 to 100° C., which is a (methyl methacrylate/acrylic acid/trifluoroethyl methacrylate) copolymer,
a second block with a Tg of less than or equal to 20° C., for example ranging from 0 to 20° C., which is a methyl acrylate homopolymer, and
an intermediate block that is a (methyl methacrylate/acrylic acid/methyl acrylate/trifluoro-ethyl methacrylate) random copolymer.

According to a third variant, the polymer according to the invention may comprise:
a first block with a Tg of greater than or equal to 40° C., for example ranging from 85 to 115° C., which is an (isobornyl acrylate/isobutyl methacrylate) copolymer,
a second block with a Tg of less than or equal to 20° C., for example ranging from −85 to −55° C., which is a 2-ethylhexyl acrylate homopolymer, and
an intermediate block that is an (isobornyl acrylate/isobutyl methacrylate/2-ethylhexyl acrylate) random copolymer.

According to a fourth variant, the polymer according to the invention may comprise:
a first block with a Tg of greater than or equal to 40° C., for example ranging from 85 to 115° C., which is an (isobornyl acrylate/methyl methacrylate) copolymer,
a second block with a Tg of less than or equal to 20° C., for example ranging from −85 to −55° C., which is a 2-ethylhexyl acrylate homopolymer, and
an intermediate block that is an (isobornyl acrylate/methyl methacrylate/2-ethylhexyl acrylate) random copolymer.

According to a fifth variant, the polymer according to the invention may comprise:
a first block with a Tg of greater than or equal to 40° C., for example ranging from 95 to 125° C., which is an (isobornyl acrylate/isobornyl methacrylate) copolymer,
a second block with a Tg of less than or equal to 20° C., for example ranging from −85 to −55° C., which is a 2-ethylhexyl acrylate homopolymer, and
an intermediate block that is an (isobornyl acrylate/isobornyl methacrylate/2-ethylhexyl acrylate) random copolymer.

According to a sixth variant, the polymer according to the invention may comprise:
a first block with a Tg of greater than or equal to 40° C., for example ranging from 85 to 115° C., which is an (isobornyl methacrylate/isobutyl methacrylate) copolymer,
a second block with a Tg of less than or equal to 20° C., for example ranging from −35 to −5° C., which is an isobutyl acrylate homopolymer, and
an intermediate block that is an (isobornyl methacrylate/isobutyl methacrylate/isobutyl acrylate) random copolymer.

According to a seventh variant, the polymer according to the invention may comprise:
a first block with a Tg of greater than or equal to 40° C., for example ranging from 95 to 125° C., which is an (isobornyl acrylate/isobornyl methacrylate) copolymer,
a second block with a Tg of less than or equal to 20° C., for example ranging from −35 to −5° C., which is an isobutyl acrylate homopolymer, and
an intermediate block that is an (isobornyl acrylate/isobornyl methacrylate/isobutyl acrylate) random copolymer.

According to an eighth variant, the polymer according to the invention may comprise:
a first block with a Tg of greater than or equal to 40° C., for example ranging from 60 to 90° C., which is an (isobornyl acrylate/isobutyl methacrylate) copolymer,
a second block with a Tg of less than or equal to 20° C., for example ranging from −35 to −5° C., which is an isobutyl acrylate homopolymer, and
an intermediate block that is an (isobornyl acrylate/isobutyl methacrylate/isobutyl acrylate) random copolymer.

One cosmetic composition that is particularly advantageous according to the invention is a cosmetic composition in which:
the ethylenic polymer comprises a first block or poly(isobornyl acrylate/methyl methacrylate) block with a Tg of 100° C., a second poly(2-ethylhexyl acrylate) block with a Tg of −70° C. and an intermediate block that is an (isobornyl acrylate/methyl methacrylate/2-ethylhexyl acrylate) random polymer; and
the tensioning agent is an aqueous dispersion of colloidal silica.

Another cosmetic composition that is particularly advantageous according to the invention is a cosmetic composition in which:
the ethylenic polymer comprises a first block or poly(isobornyl acrylate/isobornyl methacrylate) block with a Tg of 110° C., a second poly(2-ethylhexyl acrylate) block with a Tg of −70° C. and an intermediate block that is an (isobornyl acrylate/isobornyl methacrylate/2-ethylhexyl acrylate) random polymer; and
the tensioning agent is an aqueous dispersion of colloidal silica.

### SECOND EMBODIMENT

According to a second embodiment, the polymer according to the invention comprises a first block with a glass transition temperature (Tg) of between 20 and 40° C., in accordance with the blocks described in c) and a second block with a glass transition temperature of less than or equal to 20° C., as described above in b) or a glass transition temperature of greater than or equal to 40° C., as described in a) above.

Preferably, the proportion of the first block with a Tg of between 20 and 40° C. ranges from 10% to 85%, better still from 30% to 80% and even better still from 50% to 70% by weight of the polymer.

When the second block is a block with a Tg of greater than or equal to 40° C., it is preferably present in a proportion ranging from 10% to 85%, better still from 20% to 70% and even better still from 30% to 70% by weight of the polymer.

When the second block is a block with a Tg of less than or equal to 20° C., it is preferably present in a proportion ranging from 10% to 85%, better still from 20% to 70% and even better still from 20% to 50% by weight of the polymer.

Preferably, the first block with a Tg of between 20 and 40° C. is a copolymer derived from monomers which are such that the corresponding homopolymer has a Tg of greater than or equal to 40° C., and from monomers which are such that the corresponding homopolymer has a Tg of less than or equal to 20° C..

Advantageously, the second block with a Tg of less than or equal to 20° C. or with a Tg of greater than or equal to 40° C. is a homopolymer.

Thus, according to a first variant of this second embodiment, the polymer according to the invention may comprise:
a first block with a Tg of between 20 and 40° C., for example with a Tg of 25 to 39° C., which is a copolymer comprising at least one methyl acrylate monomer, at least one methyl methacrylate monomer and at least one acrylic acid monomer,
a second block with a Tg of greater than or equal to 40° C., for example ranging from 85 to 125° C., which is a homopolymer composed of 2-ethylhexyl acrylate monomers, and
an intermediate block consisting of a methyl methacrylate, acrylic acid and 2-ethylhexyl acrylate random polymer.

According to a second variant of this second embodiment, the polymer according to the invention may comprise:
a first block with a Tg of between 20 and 40° C., for example with a Tg of 21 to 39° C., which is an (isobornyl acrylate/isobutyl methacrylate/2-ethylhexyl acrylate) copolymer,
a second block with a Tg of less than or equal to 20° C., for example ranging from −65 to −35° C., which is a methyl methacrylate homopolymer, and
an intermediate block that is an (isobornyl acrylate/isobutyl methacrylate/2-ethylhexyl acrylate/methyl methacrylate) random copolymer.

According to a third variant of this second embodiment, the polymer according to the invention may comprise:
a first block with a Tg of between 20 and 40° C., for example with a Tg from 21 to 39° C., which is an (isobornyl acrylate/methyl acrylate/acrylic acid) copolymer,
a second block with a Tg of greater than or equal to 40° C., for example ranging from 85 to 115° C., which is an isobornyl acrylate homopolymer, and
an intermediate block that is an (isobornyl acrylate/methyl acrylate/acrylic acid) random copolymer.

The composition according to the invention generally comprises a fatty phase in which the polymer described above is soluble, i.e. in which it forms a molecular solution, or in which it is dispersible.

Advantageously, the ethylenic polymer as described above is present in the fatty phase.

The said fatty phase represents, for example, from 0.5% to 80% by weight, preferably from 1% to 55% by weight and better still from 1% to 25% by weight relative to the total weight of the composition.

The composition also advantageously comprises water, for example in the form of an aqueous phase, in which the tensioning agent is generally present, although it may, as a variant, be present in the fatty phase, depending on its nature.

It is understood that the said cosmetic composition will also comprise a medium compatible with the skin.

The said medium is generally cosmetically acceptable, i.e. it has a pleasant odour, colour and feel, which are compatible with a cosmetic use, and does not cause any discomfort (stinging, tautness or redness) liable to put the user off.

The fatty phase of the composition according to the invention may consist especially of fatty substances that are liquid at room temperature (25° C. in general) and/or fatty substances that are solid at room temperature, such as waxes, pasty fatty substances and gums, and mixtures thereof. These fatty substances may be of animal, plant, mineral or synthetic origin. This fatty phase may also contain lipophilic organic solvents.

As fatty substances that are liquid at room temperature, often referred to as “oils”, and can be used according to the invention, mention may be made of: hydrocarbon-based oils of animal origin such as perhydrosqualene; hydrocarbon-based plant oils such as liquid triglycerides of fatty acids of 4 to 10 carbon atoms, for instance heptanoic or octanoic acid triglycerides, or sunflower oil, maize oil, soybean oil, grapeseed oil, sesame seed oil, apricot oil, macadamia oil, castor oil, avocado oil, caprylic/capric acid triglycerides, jojoba oil or shea butter; linear or branched hydrocarbons of mineral or synthetic origin, such as isododecane, liquid paraffins and derivatives thereof, petroleum jelly, polydecenes, and hydrogenated polyisobutene such as parleam; synthetic esters and ethers, especially of fatty acids, for instance purcellin oil, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate or fatty alcohol heptanoates, octanoates and decanoates; polyol esters, for instance propylene glycol dioctanoate, neopentyl glycol diheptanoate or diethylene glycol diisononanoate; and pentaerythritol esters; fatty alcohols containing from 8 to 26 carbon atoms, for instance octyldodecanol, 2-butyloctanol, 2-hexyldecanol, 2-undecylpentadecanol or oleyl alcohol; partially hydrocarbon-based and/or silicone-based fluoro oils; silicone oils, for instance volatile or non-volatile, linear or cyclic polymethylsiloxanes (PDMSs) that are liquid or pasty at room temperature, for instance cyclomethicones, dimethicones, optionally comprising a phenyl group, for instance phenyl trimethicones, phenyltrimethylsiloxydiphenylsiloxanes, diphenylmethyldimethyltrisiloxanes, diphenyl dimethicones, phenyl dimethicones and polymethylphenylsiloxanes; and mixtures thereof.

These oils may be present in a content ranging from 0.01% to 90% and better still from 0.1% to 85% by weight, relative to the total weight of the composition.

The composition according to the invention may also contain ingredients commonly used in cosmetics, such as thickeners, sequestering agents, fragrances, acidifying or basifying agents, preserving agents, sunscreens, surfactants, fillers, pigments and dyes, and mixtures thereof.

It may also contain anti-ageing active agents with an effect complementary to the polymers defined above, such as at least one compound chosen from the desquamating agents, moisturizers, agents for stimulating keratinocyte proliferation and/or differentiation, agents for stimulating collagen and/or elastin synthesis of for preventing their degradation, depigmenting agents, anti-glycation agents, agents for stimulating glycosaminoglycan synthesis, dermo-decontracting or muscle-relaxing agents, antioxidants and free-radical scavengers, and mixtures thereof.

Needless to say, a person skilled in the art will take care to select this or these optional additional compound(s) and/or the amount thereof, such that the advantageous properties of the corresponding composition according to the invention are not, or are not substantially, adversely affected by the envisaged addition.

The composition according to the invention may especially be in the form of a suspension, a dispersion, a solution, a gel, an emulsion, especially an oil-in-water (O/W) or water-in-oil (W/O) emulsion, or a multiple emulsion (W/O/W or polyol/O/W or O/W/O), in the form of a cream, a paste, a mousse, a dispersion of vesicles, especially of ionic or nonionic lipids, or a two-phase or multi-phase lotion.

A person skilled in the art may select the appropriate presentation form, and also the method for preparing it, on the basis of his general knowledge, taking into account firstly the nature of the constituents used, especially their solubility in the support, and secondly the intended use of the composition.

According to a second subject, the present invention relates to the use of an ethylenic polymer as defined above to improve the remanence of the tensioning effect afforded by a tensioning agent.

The remanence of the tensioning effect is quantified by a test featured in the experimental section of this description.

According to a third subject, the present invention relates to the use of an ethylenic polymer as defined above in a cosmetic composition comprising an aqueous dispersion of mineral colloidal particles, in particular of silica, to prevent bleaching of the skin.

Finally, according to a fourth subject, the present invention relates to a cosmetic process for treating wrinkled skin intended to reduce the wrinkles and/or the small wrinkles of the skin, comprising a step consisting in applying to the said skin a composition as defined above.

The application is performed according to the usual techniques, for example by applying creams, gels, sera or lotions to the skin. intended to be treated, in particular the skin around the eyes. In the context of this process, the composition may be, for example, a care composition or a makeup composition, in particular a foundation.

The invention will now be described with reference to the following examples, which are given as non-limiting illustrations.

### DETAILED DESCRIPTION OF PARTICULAR EMBODIMENTS

Various polymers and formulations were prepared, incorporating polymers as defined above in combination with tensioning agents, and tested in order to demonstrate the improvement in the remanence of the tensioning effect induced by using such polymers in these compositions.

Before proceeding to the detailed description of the preparations of polymers and of formulations, a protocol for quantifying the remanence of the tensioning effect induced by the abovementioned polymer/tensioning agent combination will be described.
Protocol for quantifying the remanence of the tensioning effect
   Principle of the test

The desired remanence properties are achieved by means of introducing compounds acting as reinforcers, these compounds being the ethylenic polymers as defined above. The reinforcing potential of the compounds used was quantified by measuring the breaking strength of the materials (in the present case an anti-wrinkle cream).

The test consists in subjecting to compression up to the breaking point the material deposited on the surface of a flexible, deformable foam. Using this foam support makes it possible to apply a large strain to the material deposited on the surface and thus to quantify its breaking strength. The mechanical compressive stress is exerted using a cylindrical punch 1 mm in diameter; the travelling speed of the punch is 0.1 mm/s. The test is performed using a TA-XT2i texture analyser sold by the company Stable Micro System. A curve of the force F (in N) as a function of the displacement d (in mm) is thus observed, from which it is possible to determine the breaking point of the material:

[image]

### Example of Curve of Force as a Function of the Displacement

Two parameters are used to quantify the breaking strength of the material:

(1) F_{break} (N) : breaking force

(2) W_{break} (J/m²): breaking energy: area under the curve F=f(d)/area of the punch

The substrate consists of a neoprene foam 13 mm thick. The material (anti-wrinkle composition) is placed on this substrate so as to obtain, after drying for 24 hours, a film from 15 to 30 μm thick. The depositions were made using a film spreader that deposits a film of 650 μm while wet (i.e. before drying).

### Comparative Example

This example illustrates a cosmetic composition comprising a tensioning agent in the form of an aqueous dispersion of colloidal silica (Cosmo S40), the said composition being free of ethylenic polymer in accordance with the present invention.

The composition is as follows:

| Constituents | Amount | |
|---|---|---|
| Glyceryl stearate and PEG-100 stearate | 2 | g |
| Dimyristyl tartrate and cetearyl alcohol | 1.50 | g |
| and C₁₂-C₁₅-pareth-7 and | | |
| PPG-25-laureth-25 | | |
| Cyclohexasiloxane | 10 | g |
| Stearyl alcohol | 1 | g |
| Water | 66.75 | g |
| Phenoxyethanol | 1 | g |
| Sequestering agent | 0.05 | g |
| Polyacrylamide (Hostacerin AMPS from | 0.40 | g |
| Clariant) | | |
| Xanthan gum | 0.20 | g |
| Cosmo S40 (aqueous dispersion of colloidal | 17.10 | g |
| silica) | | |

The composition is prepared in the following manner:

The phase consisting of the water, the phenoxyethanol, the sequestering agent and the xanthan gum is heated to 75° C. The thickening polymer (i.e. the polyacrylamide) is then incorporated therein. The mixture is stirred until a homogeneous gel is obtained.

The phase consisting of the glyceryl stearate, the PEG-100 stearate, the dimyristyl tartrate, the cetearyl alcohol, the C₁₂-C₁₅-pareth-7, the PPG-25-laureth-25, the cyclohexasiloxane and the stearyl alcohol is heated to 75° C.. This phase is then incorporated into the preceding phase to produce an emulsion. The aqueous dispersion of colloidal silica is then incorporated into the emulsion at 40-45° C. and stirring is continued until the emulsion has completely cooled.

### EXAMPLE 1

This example illustrates the preparation of a poly(isobornyl acrylate/methyl methacrylate/2-ethylhexyl acrylate) polymer and of a composition comprising this polymer in combination with a tensioning agent in the form of an aqueous dispersion of colloidal silica.
Preparation of poly(isobornyl acrylate/methyl methacrylate/2-ethylhexyl acrylate)

100 g of isododecane are introduced into a 1 litre reactor and the temperature is then increased so as to pass from room temperature (25° C.) to 90° C. over 1 hour.

150 g of isobornyl acrylate, 60 g of methyl methacrylate, 110 g of isododecane and 1.8 g of 2,5-bis(2-ethylhexanoylperoxy)-2,5-dimethylhexane (Trigonox® 141 from Akzo Nobel) are then added, at 90° C. and over 1 hour.

The mixture is maintained at 90° C. for 1 hour 30 minutes.

90 g of 2-ethylhexyl acrylate, 90 g of isododecane and 1.2 g of 2,5-bis(2-ethylhexanoylperoxy)-2,5-dimethylhexane are then introduced into the above mixture, still at 90° C. and over 1 hour.

The mixture is maintained at 90° C. for 3 hours and is then cooled.

A solution containing 50% polymer active material in isododecane is obtained.

A polymer comprising a first block or poly(isobornyl acrylate/methyl methacrylate) block with a Tg of 100° C., a second poly(2-ethylhexyl acrylate) block with a Tg of −70° C. and an intermediate block that is an (isobornyl acrylate/methyl methacrylate/2-ethyl-hexyl acrylate) random polymer is obtained.

This polymer has a weight-average mass of 89 100 g/mol and a number-average mass of 21 300, i.e. a polydispersity index I of 4.19.
Preparation of the composition

The composition is as follows:

| Constituents | Amount | |
|---|---|---|
| Glyceryl stearate and PEG-100 stearate | 2 | g |
| Dimyristyl tartrate and cetearyl alcohol and | 1.50 | g |
| C₁₂-C₁₅-pareth-7 and PPG-25-laureth-25 | | |
| Cyclohexasiloxane | 5 | g |
| Stearyl alcohol | 1 | g |
| Water | 66.75 | g |
| Phenoxyethanol | 1 | g |
| Sequestering agent | 0.05 | g |
| Polyacrylamide (Hostacerin AMPS from | 0.40 | g |
| Clariant) | | |
| Xanthan gum | 0.20 | g |
| Cosmo S40 (aqueous dispersion of colloidal | 17.10 | g |
| silica) | | |
| Polymer prepared above | 5 | g |

The composition of this example is prepared in the same manner as that of the comparative example above, this preparation also comprising the incorporation of the polymer prepared above at 40-45° C. into the emulsion after the introduction of the aqueous dispersion of colloidal silica.

### EXAMPLE 2

This example illustrates the preparation of a poly(isobornyl acrylate/isobornyl methacrylate/2-ethylhexyl acrylate) polymer and of a composition comprising this polymer in combination with a tensioning agent in the form of an aqueous dispersion of colloidal silica.
Preparation of poly(isobornyl acrylate/isobornyl methacrylate/2-ethylhexyl acrylate)

100 g of isododecane are introduced into a 1 litre reactor and the temperature is then increased so as to pass from room temperature (25° C.) to 90° C. over 1 hour.

105 g of isobornyl acrylate, 105 g of isobornyl methacrylate, 110 g of isododecane and 1.8 g of 2, 5-bis(2-ethylhexanoylperoxy)-2, 5-dimethylhexane (Trigonox® 141 from Akzo Nobel) are then added, at 90° C. and over 1 hour.

The mixture is maintained at 90° C. for 1 hour 30 minutes.

90 g of 2-ethylhexyl acrylate, 90 g of isododecane and 1.2 g of 2,5-bis(2-ethylhexanoyl-peroxy)-2,5-dimethylhexane are then introduced into the above mixture, still at 90° C. and over 1 hour.

The mixture is maintained at 90° C. for 3 hours and is then cooled.

A solution containing 50% polymer active material in isododecane is obtained.

A polymer comprising a first block or poly(isobornyl acrylate/isobornyl methacrylate) block with a Tg of 110° C., a second poly-2-ethylhexyl acrylate block with a Tg of −70° C. and an intermediate block that is an (isobornyl acrylate/isobornyl methacrylate/2-ethylhexyl acrylate) random polymer is obtained.

This polymer has a weight-average mass of 103 900 g/mol and a number-average mass of 21 300, i.e. a polydispersity index I of 4.89.
Preparation of the composition

The composition is as follows:

| Constituents | Amount | |
|---|---|---|
| Glyceryl stearate and PEG-100 stearate | 2 | g |
| Dimyristyl tartrate and cetearyl alcohol and | 1.50 | g |
| C₁₂-C₁₅-pareth-7 and PPG-25-laureth-25 | | |
| Cyclohexasiloxane | 5 | g |
| Stearyl alcohol | 1 | g |
| Water | 66.75 | g |
| Phenoxyethanol | 1 | g |
| Sequestering agent | 0.05 | g |
| Polyacrylamide (Hostacerin AMPS from | 0.40 | g |
| Clariant) | | |
| Xanthan gum | 0.20 | g |
| Cosmo S40 (aqueous dispersion of colloidal | 17.10 | g |
| silica) | | |
| Polymer prepared above | 5 | g |

The composition of this example is prepared in the same manner as that of the comparative example above, this preparation also comprising the incorporation of the polymer prepared above at 40-45° C. into the emulsion after the introduction of the aqueous dispersion of colloidal silica.

### EXAMPLE 3

Demonstration of the Remanent Tensioning Effect

The protocol for quantifying the remanence of the tensioning effect was used for the three compositions of the comparative example and Examples 1 and 2.

This protocol is directed towards quantifying the reinforcing potential of the polymers of Example 1 and of Example 2 (in isododecane) when introduced into an anti-wrinkle composition.

| | | W_{break} |
|---|---|---|
| Composition | F_{break} (N) | (J/m²) |
| Comparative example | 0.18 ± 0.02 | 19 ± 3 |
| Example 1 (7% CS40 silica + 2.5% | 0.29 ± 0.01 | 83 ± 9 |
| polymer) | | |
| Example 2 (7% CS40 silica + 2.5% | 0.34 ± 0.01 | 98 ± 5 |
| polymer) | | |

These results demonstrate the reinforcing role of the two polymers studied in the presence of a tensioning agent. This reinforcing role is illustrated by an increase in the breaking force and the breaking energy.

### EXAMPLE 4

Effect on the Bleaching of the Skin

The cosmetic compositions corresponding to the comparative example and to Example 2 above were spread using a mechanical film spreader onto a contrast card (Pruifkarte type 24/5-250 cm²) sold by the company Erichsen (film thickness: 30 μm). The compositions were then dried for three hours at a temperature of 20° C. and photographs of the treated areas were taken.

The appearance of unattractive white deposits on the treated area were noted in the case of the composition of the comparative example. In the case of the composition of Example 2 according to the invention, such unattractive deposits are absent.

### REFERENCES CITED

[1] FR-A-2 758 083;
[2] U.S. -6,139,322;
[3] U.S. Pat. No. -6,465,001;
[4] U.S. Pat. No. -5,349,003;
[5] EP-1 038 519;
[6] FR-2 819 429;
[7] Food Gels, Peter Harris, Elsevier 1989, Chapter 3;
[8] Food Gels, Peter Harris, Elsevier 1989, Chapter 1;
[9] Food Gels, Peter Harris, Elsevier 1989, Chapter 6;
[10] FR-2 829 025;
[11] Kirk-Othmer Encyclopedia of Chemical Technology, 3rd edition, volume 21, pp. 492-507, Wiley Interscience, 1983.

1. A cosmetic composition adapted for a topical application on the skin comprising, in a medium compatible with the skin:
at least one tensioning agent, the sad tensioning agent being present in a content ranging from 0.01% to 20% relative to the total weight of the composition; and
at least one non-elastomeric, water-insoluble film-forming linear block ethylenic polymer, the polymer being present in a content ranging from 0.01% to 20% relative to the total weight of the composition.

2. The cosmetic composition according to claim 1, the composition being an anti-wrinkle composition.

3. Cosmetic The cosmetic composition according to claim 1, in which the tensioning agent is an agent that produces, at a concentration of 7% in water, a shrinkage of isolated stratum corneum, measured using an extensometer, of more than 1% at 30° C. under a relative humidity of 40%.

4. The cosmetic composition according to claim 1, in which the tensioning agent is present in the composition in a content ranging from 1% to 10% relative to the total weight of the composition.

5. The cosmetic composition according to claim 1, in which the tensioning agent is chosen from synthetic polymers, polymers of natural origin, mixed silicates, wax microparticles and colloidal particles of mineral fillers, and mixtures thereof.

6. The cosmetic composition according to claim 5, in which the synthetic polymers are chosen from polyurethane polymers and copolymers, acrylic polymers and copolymers, sulfonated isophthalic acid polymers, grafted silicone polymers, water-soluble or water-dispersible polymers comprising water-soluble or water-dispersible units and units with an LCST, and mixtures thereof.

7. The cosmetic composition according to claim 5, in which the polymers of natural origin are chosen from plant proteins and plant protein hydrolysates, polysaccharides of plant origin optionally in the form of microgels, and latices of plant origin, and mixtures thereof.

8. The cosmetic composition according to claim 1, in which the ethylenic polymer is present in a content ranging from 1% to 10% relative to the total weight of the composition.

9. The cosmetic composition according to claim 1, in which the block ethylenic polymer comprises at least one first block and at least one second block that are mutually incompatible and that have different glass transition temperatures (Tg), the first and second blocks being connected together via an intermediate segment comprising at least one constituent monomer of the first block and at least one constituent monomer of the second block, the polymer having a polydispersity index I of greater than 2.

10. The cosmetic composition according to claim 9, in which the polymer has a polydispersity index of greater than or equal to 2.5.

11. The cosmetic composition according to claim 10, in which the polymer has a polydispersity index ranging from 2.8 to 6.

12. The cosmetic composition according to any one of the preceding claims claim 10, in which the polymer has a weight-average mass (Mw) of less than or equal to 300 000.

13. The cosmetic composition according to any one of the preceding claims claim 1, in which the polymer has a weight-average mass (Mw) ranging from 35 000 to 200 000.

14. The cosmetic composition according to claim 1, in which the polymer has a number-average mass (Mn) of less than or equal to 70 000.

15. The cosmetic composition according to claim 1, in which the polymer has a number-average mass (Mn) ranging from 10 000 to 60 000.

16. The cosmetic composition according to claim 9, in which the difference in temperature between the glass transition temperatures (Tg) of the first and second blocks is greater than 20° C.

17. The cosmetic composition according to claim 9, in which the first block is chosen from:
a) a block with a Tg of greater than or equal to 40° C.,
b) a block with a Tg of less than or equal to 20° C.,
c) a block with a Tg of between 20 and 40° C., and the second block being chosen from a category a), b) or c) different from the first block.

18. The cosmetic composition according to claim 17, in which the block with a Tg of greater than or equal to 40° C. has a Tg ranging from 40° C. to 150° C.

19. The cosmetic composition according to claim 17, in which the block with a Tg of greater than or equal to 40° C. is a homopolymer or a copolymer.

20. The cosmetic composition according to claim 19, in which the block with a Tg of greater than or equal to 40° C., when it is a homopolymer, is derived from monomers which are such that the homopolymers prepared from these monomers have glass transition temperatures of greater than or equal to 40° C.

21. The cosmetic composition according to claim 19, in which the block with a Tg of greater than or equal to 40° C., when it is a copolymer, is totally or partially derived from one or more monomers, the nature and concentration of which are chosen such that the Tg of the resulting copolymer is greater than or equal to 40° C.

22. The cosmetic composition according to claim 20, in which the monomers whose homopolymers have glass transition temperatures of greater than or equal to 40° C. are chosen from the following monomers:
methacrylates of formula
CH₂═C(CH₃)—COOR₁
in which R₁ represents a linear or branched unsubstituted alkyl group containing from 1 to 4 carbon atoms, or R₁ represents a C₄ to C₁₂ cycloalkyl group,
acrylates of formula CH₂═CH—COOR₂ in which R₂ represents a C₄ to C₁₂ cycloalkyl group
(meth)acrylamides of formula: 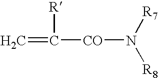
in which R₇ and R₈, which may be identical or different, each represent a hydrogen atom or a linear or branched C₁ to C₁₂ alkyl group; or R₇ represents H and R₈ represents a 1,1-dimethyl-3-oxobutyl group, and R′ denotes H or methyl;
and mixtures thereof.

23. The cosmetic composition according to claim 22, in which the monomers whose homopolymers have glass transition temperatures of greater than or equal to 40° C. are chosen from methyl methacrylate, isobutyl (meth)acrylate, isobornyl (meth)acrylate, and mixtures thereof.

24. The cosmetic composition according to claim 17, in which the block with a Tg of less than or equal to 20° C. is a homopolymer or a copolymer.

25. The cosmetic composition according to claim 24, in which the block with a Tg of less than or equal to 20° C., when it is a homopolymer, is derived from monomers which are such that the homopolymers prepared from these monomers have glass transition temperatures of less than or equal to 20° C.

26. The cosmetic composition according to claim 24, in which the block with a Tg of less than or equal to 20° C., when it is a copolymer, is totally or partially derived from one or more monomers, the nature and concentration of which are chosen such that the Tg of the resulting copolymer is less than or equal to 20° C.

27. The cosmetic composition according to claim 24, in which the monomers whose homopolymers have glass transition temperatures of less than or equal to 20° C. are chosen from the following monomers:
acrylates of formula CH₂═CHCOOR₃, R₃ representing a linear or branched C₁ to C₁₂ alkyl group, with the exception of the tert-butyl group, in which one or more hetero atoms chosen from O, N and S is (are) optionally intercalated, the alkyl group also possibly being optionally substituted with one or more substituerts chosen from hydroxyl groups and halogen atoms (Cl, Br, I and F), or R₃ represents an (alkyl group C₁-C₁₂)-O-POE (POE denoting polyoxyethylene with repetition of the oxyethylene group from 5 to 30 times), or R₃ represents a polyoxyethylene group comprising from 5 to 30 ethylene oxide units;
methacrylates of formula CH₂═C(CH₃)—COOR4, R4 representing a linear or branched C₆ to C₁₂ alkyl group, in which one or more hetero atoms chosen from O, N and S is (are) optionally intercalated, the alkyl group also possibly being optionally substituted with one or more substituents chosen from hydroxyl groups and halogen atoms (Cl, Br, I or F);
vinyl esters of formula R₅-CO-O-CH═CH₂ in which R₅ represents a linear or branched C₄ to C₁₂ alkyl group;
C₄ to C₁₂ alkyl vinyl ethers;
N-(C₄ to C₁₂-alkyl) acrylamides;
and mixtures thereof.

28. The cosmetic composition according to claim 27, in which the monomers whose homopolymers have glass transition temperatures of less than or equal to 20° C. are chosen from alkyl acrylates in which the alkyl chain contains from 1 to 10 carbon atoms, with the exception of the tert-butyl group.

29. The cosmetic composition according to claim 17, in which the block with a Tg of between 20 and 40° C. is a homopolymer or a copolymer.

30. The cosmetic composition according to claim 29, in which the block with a Tg of between 20 and 40° C., when it is a homopolymer, is derived from monomers (or main monomers) which are such that the homopolymers prepared from these monomers have glass transition temperatures of between 20 and 40° C.

31. The cosmetic composition according to claim 29, in which the block with a Tg of between 20 and 40° C., when it is a copolymer, is totally or partially derived from one or more monomers (or main monomers), the nature and concentration of which are chosen such that the Tg of the resulting copolymer is between 20 and 40° C.

32. The cosmetic composition according to claim 29, in which the monomers whose homopolymer has a glass transition temperature of between 20 and 40° C. are chosen from n-butyl methacrylate, cyclodecyl acrylate, neopentyl acrylate and isodecylacrylamide, and mixtures thereof.

33. The cosmetic composition according to claim 17, in which the first block and/or the second block comprises at least one additional monomer.

34. The cosmetic composition according to claim 33, in which the additional monomer is chosen from hydrophilic monomers and ethylenically unsaturated monomers comprising one or more silicon atoms, and mixtures thereof.

35. The cosmetic composition according to claim 34, in which the hydrophilic monomer is chosen from:
ethylenically unsaturated monomers comprising at least one carboxylic or sulfonic acid function;
ethylenically unsaturated monomers comprising at least one hydroxyl function;
ethylenically unsaturated monomers comprising at least one tertiary amine function;
methacrylates of formula CH₂═C(CH₃)—COOR6 in which R6 represents a linear or branched alkyl group containing from 1 to 4 carbon atoms, the alkyl group being substituted with one or more substituents chosen from hydroxyl groups and halogen atoms (Cl, Br, I or F);
methacrylates of formula CH₂═C(CH₃)—COOR₉, R₉ representing a linear or branched C₆ to C₁₂ alkyl group in which one or more hetero atoms chosen from O, N and S is (are) optionally intercalated, the said alkyl group being substituted with one or more substituents chosen from hydroxyl groups and halogen atoms (Cl, Br, I or F);
acrylates of formula CH₂═CHCOOR₁₀, R₁₀ representing a linear or branched C₁, to C₁₂ alkyl group substituted with one or more substituents chosen from hydroxyl groups and halogen atoms (Cl, Br, I or F), or R₁₀ represents a (C₁ to C₁₂ alkyl)-O-POE (POE denoting polyoxyethylene with repetition of the oxyethylene unit 5 to 30 times), or R₁₀ represents a polyoxyethylenated group comprising from 5 to 30 ethylene oxide units.

36. The cosmetic composition according to claim 17, in which the block ethylenic polymer comprises a first block with a Tg of greater than or equal to 40° C. and a second block with a Tg of less than or equal to 20° C.

37. The cosmetic composition according to claim 36, in which the first block with a Tg of greater than or equal to 40° C. is a copolymer derived from monomers which are such that the homopolymer prepared from these monomers has a glass transition temperature of greater than or equal to 40° C.

38. The cosmetic composition according to claim 36, in which the second block with a Tg of less than or equal to 20° C. is a homopolymer derived from monomers which are such that the homopolymer prepared from these monomers has a glass transition temperature of less than or equal to 20° C.

39. The cosmetic composition according to claim 36, in which the proportion of the block with a Tg of greater than or equal to 40° C. ranges from 20% to 90%, by weight of the polymer.

40. The cosmetic composition according to claim 36, in which the proportion of the block with a Tg of less than or equal to 20° C. ranges from 5% to 75%, by weight of the polymer.

41. The cosmetic composition according to claim 36, in which the polymer comprises:
a first block with a Tg of greater than or equal to 40° C., which is a (methyl methacrylate/acrylic acid) copolymer,
a second block with a Tg of less than or equal to 20° C., which is a methyl acrylate homopolymer, and
an intermediate block that is a (methyl methacrylate/acrylic acid/methyl acrylate) copolymer.

42. The cosmetic composition according to claim 36, in which the polymer comprises:
a first block with a Tg of greater than or equal to 40° C., which is a (methyl methacrylate/acrylic acid/trifluoroethyl methacrylate) copolymer,
a second block with a Tg of less than or equal to 20° C., which is a methyl acrylate homopolymer, and
an intermediate block that is a (methyl methacrylate/acrylic acid/methyl acrylate/trifluoroethyl methacrylate) random copolymer.

43. The cosmetic composition according to claim 36, in which the polymer comprises:
a first block with a Tg of greater than or equal to 40° C., which is a an (isobornyl acrylate/isobutyl methacrylate) copolymer,
a second block with a Tg of less than or equal to 20° C., which is a 2-ethylhexyl acrylate homopolymer, and
an intermediate block that is an (isobomyl acrylate/isobutyl methacrylate/2-ethylhexyl acrylate) random copolymer.

44. The cosmetic composition according to claim 36, in which the polymer comprises:
a first block with a Tg of greater than or equal to 40° C., which is gan (isobornyl acrylate/methyl methacrylate) copolymer,
a second block with a Tg of less than or equal to 20° C., which is a 2-ethylhexyl acrylate homopolymer, and
an intermediate block that is an (isobornyl acrylate/methyl methacrylate/2-ethylhexyl acrylate) random copolymer.

45. The cosmetic composition according to claim 36, in which the polymer comprises:
a first block with a Tg of greater than or equal to 40° C., which is an (isobornyl acrylate/isobornyl methacrylate) copolymer,
a second block with a Tg of less than or equal to 20° C., which is a 2-ethylhexyl acrylate homopolymer, and
an intermediate block that is an (isobornyl acrylate/isobornyl methacrylate/2-ethylhexyl acrylate) random copolymer.

46. Cosmetic The cosmetic composition according to claim 36, in which the polymer comprises:
a first block with a Tg of greater than or equal to 40° C., which is an (isobornyl methacrylate/isobutyl methacrylate) copolymer,
a second block with a Tg of less than or equal to 20° C., which is an isobutyl acrylate homopolymer, and
an intermediate block that is an (isobornyl methacrylate/isobutyl methacrylate/isobutyl acrylate) random copolymer.

47. The cosmetic composition according to claim 36, in which the polymer comprises:
a first block with a Tg of greater than or equal to 40° C., which is an (isobornyl acrylate/isobornyl methacrylate) copolymer,
a second block with a Tg of less than or equal to 20° C., which is an isobutyl acrylate homopolymer, and
an intermediate block that is an (isobornyl acrylate/isobornyl methacrylate/isobutyl acrylate) random copolymer.

48. The cosmetic composition according to claim 36, in which the polymer comprises:
a first block with a Tg of greater than or equal to 40° C., which is an (isobornyl acrylate/isobutyl methacrylate) copolymer,
a second block with a Tg of less than or equal to 20° C., which is an isobutyl acrylate homopolymer, and
an intermediate block that is an (isobornyl acrylate/isobutyl methacrylate/isobutyl acrylate) random copolymer.

49. The cosmetic composition according to claim 36, in which:
the ethylenic polymer comprises a first block or poly(isobornyl acrylate/methyl methacrylate) block with a Tg of 100° C., a second poly(2-ethylhexyl acrylate) block with a Tg of −70° C. and an intermediate block that is an (isobornyl acrylate/methyl methacrylate/2-ethylhexyl acrylate) random polymer; and
the tensioning agent is an aqueous dispersion of colloidal silica.

50. The cosmetic composition according to claim 36, in which:
the ethylenic polymer comprises a first block or poly(isobornyl acrylate/isobornyl methacrylate) block with a Tg of 110° C., a second poly(2-ethylhexyl acrylate) block with a Tg of −70° C. and an intermediate block that is an (isobornyl acrylate/isobornyl methacrylate/2-ethylhexyl acrylate) random polymer;
the tensioning agent is an aqueous dispersion of colloidal silica.

51. The cosmetic composition according to claim 1, in which the sad composition comprises a fatty phase.

52. The cosmetic composition according to claim 51, in which the block ethylenic polymer is present in the fatty phase.

53. The cosmetic composition according to claim 1, in which the composition comprises an aqueous phase.

54. The cosmetic composition according to claim 1, which is in the form of an emulsion.

55. A process of improving the remanence of a tensioning effect afforded by the tensioning agent as defined according to claim 1, comprising adding an ethylenic polymer as defined according to claim 1 to the tensioning agent.

56. A cosmetic process of preventing bleaching of skin comprising applying a cosmetic composition comprising an aqueous dispersion of mineral colloidal particles-and an ethylenic polymer as defined according to claim 1 to the skin.

57. A cosmetic process for treating wrinkled skin intended to reduce the wrinkles and/or the small wrinkles of the skin, comprising a step consisting in applying to the skin a composition as defined according to claim 1.

58. The cosmetic process according to claim 57, in which the composition is applied to the contour of the eyes.

59. The cosmetic process according to claim 57, in which the composition is a care composition or a makeup composition.
